(19) United States
(12) Patent Application Publication
(10) Pub. No.: US 2009/0281625 A1
(43) Pub. Date: Nov. 12, 2009

(54) EXPANDABLE INTERVERTEBRAL IMPLANT

(75) Inventor: Albert Enayati, Los Angeles, CA (US)

Correspondence Address: KRAMER & AMADO, P.C., 1725 DUKE STREET, SUITE 240, ALEXANDRIA, VA, 22314, US

(73) Assignee: RHAUSLER, Inc., Redwood City, CA (US)

(21) Appl. No.: 12/115,600

(22) Filed: May 6, 2008

(51) Int. Cl. A61F 2/44

(52) U.S. Cl. 623/17.11

(57) ABSTRACT

An expandable intervertebral implant includes a bone graft implant dimensioned for insertion within an intervertebral space defined between adjacent vertebrae, able to vertically elevate and expand a plurality of ribs into the surrounding bone. The expandable intervertebral implant has a tubular outer body portion having a cylindrical axial bore with a triangular or elliptical cross-section and a plurality of ribs disposed on its outer body dimensioned to fit snugly within the space and an expansion cylinder with a triangular or elliptical cross-section slidably mounted within the axial bore of the tubular outer body. The tubular outer body portion of the expandable intervertebral implant permits the expansion and retraction of the ribs into or out of the surrounding bone as the expansion cylinder rotates.

### BACKGROUND OF THE INVENTION

1. Field of the Invention

The present invention relates to an osteogenic interbody fusion implant device and, more particularly, to a non-threaded intervertebral bone implant having a plurality of expandable ribs configured to facilitate securement of the implant within the intervertebral space.

2. Description of the Related Art

In chordate animals having backbones, the spine is a flexible column formed of a plurality of bones called vertebrae. The vertebrae are hollow and piled one upon the other, forming a strong, hollow column for support of the cranium and trunk. The hollow core of the spine houses and protects the nerves of the spinal cord. The different vertebrae are connected to one another by means of articular processes and intervertebral, fibro-cartilaginous bodies.

These intervertebral fibro-cartilaginous bodies, also known as intervertebral disks, are made of fibrous rings filled with pulpy material, known as the nucleus pulposus. These disks function as spinal shock absorbers and also cooperate with the synovial joints to facilitate movement and maintain flexibility of the spine. When one or more disks degenerate through accident or disease, nerves passing near the affected area may be compressed and are consequently irritated. The result may be chronic and/or debilitating back pain. Various methods and devices, both surgical and non-surgical, have been designed to relieve such back pain.

One method, interbody fusion, involves stretching the spine into a natural position so that nerve root canal sizes increase, thereby eliminating or reducing nerve irritation. The space between vertebrae is maintained by fusing the vertebrae in the affected area together at a fixed distance. Numerous prosthetic implants have been suggested to fill the voids between vertebrae. For example, a spherical cage implant made of either metal or ceramic material may be inserted between adjacent vertebrae. This cage may have an interior cavity within which bone fragment are inserted. Such bone fragments may be autogenic and are intended to promote subsequent bone growth and fusion of the vertebrae.

Another method of preventing contact of vertebrae may involve longitudinal insertion of a stud between two vertebrae. Springs may connect upper and lower disks to form an artificial intervertebral disk. The artificial disk may be held between adjacent vertebrae by spikes which project from the disk into the surfaced of the vertebrae. Prongs or screws may secure a rigid, porous plug after it is inserted between vertebrae. The porous nature of the plug may be osteogenic, facilitating the growth of bone tissue.

An implant able bone plug may be inserted between vertebrae. In one embodiment, the exterior of the plug may have external threading which will, when the plug is rotated, advance the plug into prepared sites between the vertebrae. A portion of the plug may have a slot designed to receive the end of a key, which is used to rotate the plug. Aborted hole maybe formed between two adjacent vertebrae, allowing a graft medium such as finely-chopped, cortical or cancellous bone chips to be poured into the bored hole.

A substantially open fission cage may be inserted between the opposing bony surfaces of adjacent vertebrae by screwing the cage into place. The cage may be filled with bone chips or other osteogenic substances. When inserted into the intervertebral space, intimate contact between the bone inducing substance contained within the cage and the native bone may occur through the outer surface of the cage.

Ideally, a fusion graft should stabilize the intervertebral space and become fused to adjacent vertebrae. During the time it takes for fusion to occur, the graft should have sufficient structural integrity to withstand the stress of maintaining the space without substantially degrading or deforming and have sufficient stability to remain securely in place prior to actual fusion. Consequently, a fusion graft should contain some kind of anchor and a bone inducing substance, causing rapid bone growth and quick fusion of the graft to adjacent vertebrae. In addition, the material from which the fusion graft is made should be biocompatible. Further, the implant material should closely resemble host tissue and not elicit an immune response from the host.

All of the above-described implants are intended to support and maintain an appropriate intervertebral space. However, many implants are made of metals and ceramic materials and, while biocompatible, do not precisely mimic the body's natural bone tissue. A graft in the form of bone chips may eventually result in fusion between the vertebrae. If adequate fusion of the bone chips occurs, the final fused graft may closely mimic the body's naturally occurring tissues. However, when the bone chips are inserted, they may not remain contained between the vertebrae for a sufficient time to adequately fuse to each other and to adjacent vertebrae. A bone plug may have a threaded outer surface to assist in placement of the implant between the adjacent vertebrae. The external threads, however, compromise the strength of the implant. In addition, the threaded bone implant may have a tendency to back out of the prepared bore.

Other bone-engaging substrate fastening means employ several straight or curved cantilevered ribs, which may be elastically deformed to permit insertion into a hole drilled in a bone. These fasteners are well known in medical applications wherein the need for high holding strength had led to the development of anchors having multiple cantilevered ribs. In each case, the body, the attachment means, and the bone-engaging means mechanically cooperate with one another to fasten a suture, bone portion, soft tissue, implant, post, or other substrate to a bone.

There remains a need for improved intervertebral fusion implants with anchoring means, which more closely embody the ideal properties of a spinal fusion implant. There further remains a need for an expandable, intervertebral implant for facilitating arthrodesis in the disk space between adjacent vertebrae with predictable and controllable initial anchorage strength sufficient to permit gradual load sharing and provide fill repair and restoration of function during bone fusion.

The foregoing objects and advantages of the invention are illustrative of those that can be achieved by the various exemplary embodiments and are not intended to be exhaustive or limiting of the possible advantages which can be realized. Thus, these and other objects and advantages of the various exemplary embodiments will be apparent from the description herein or can be learned from practicing the various exemplary embodiments, both as embodied herein or as modified in view of any variation which may be apparent to those skilled in the art. Accordingly, the present invention resides in the novel methods, arrangements, combinations, and, improvements herein shown and described in various exemplary embodiments.

### SUMMARY OF THE INVENTION

In light of the present need for an expandable, intervertebral implant that more closely embodies the ideal properties of a spinal fusion implant, a brief summary of various exemplary embodiments is presented. Some simplifications and omissions may be made in the following summary, which is intended: to highlight and introduce some aspects of the various exemplary embodiments, but not to limit its scope. Detailed descriptions of preferred exemplary embodiments adequate to allow those of ordinary skill in the art to make and use the inventive concepts will follow in later sections.

An expandable, intervertebral implant in accordance with the present invention comprises a single, tubular, outer-body portion having at least one aperture, at least one low profile rib on its outer cylindrical surface, and an axial bore. The low profile rib is elastically deformable. The aperture extends inwardly to an axial bore containing a bone graft material.

In operation, a hole is drilled between adjacent vertebrae. The tubular, outer-body portion is inserted into the hole and advanced. As the implant advances, the low profile rib bends, contacting the surface of the implant. When the implant is fully inserted into the hole, the elastically deformable, low profile rib is driven into the surrounding bone, thereby anchoring the implant within the hole. The aperture in the surface of the tubular outer body permits ingrowth of bone into the bone graft material housed within the axial bore, thereby promoting fusion of the adjacent vertebrae.

In another embodiment, the intervertebral implant comprises a tubular, outer-body portion having a proximal end, a distal end, and an elongate body portion with a first axial bore there between. The tubular, outer-body portion has a generally cylindrical first outer surface and a cylindrical bore with a triangular or elliptical cross-section when viewed in the direction of the second axial bore. The first outer surface has at least one first aperture.

An elongate expansion cylinder is slidably disposed within the first axial bore. This cylinder has a second axial bore and a second outer surface with a triangular or elliptical cross-section when viewed in the direction of the second axial bore. The second outer surface has at least one second aperture.

Bone graft material is disposed within the second axial bore. At least one elastically-deformable, high profile rib is disposed on the first outer surface. When the expansion cylinder rotates within the first axial bore, this rib may be deformed from its normal configuration. Instead of projecting outwardly, the rib dislodges from its housing and extends away from the first outer surface, penetrating into the surrounding bone. There may be at least one conduit between the second outer surface and the second axial bore.

In operation, a hole is drilled between adjacent vertebrae and the intervertebral implant is inserted into the hole. The elongate expansion cylinder then rotates, driving the high profile rib into the surrounding bone. This anchors the implant within the intervertebral space.

In another embodiment, the tubular, outer-body portion comprises at least one frangible cylinder having at least one longitudinal slit. The frangible, tubular, outer-body portion of this cylinder has an axial bore. At least one elastically-deformable, low profile rib is located on each cylinder's outer surface. An expansion cylinder having at least one longitudinal flange or ridge on its outer surface is rotatably disposed within the axial bore. The longitudinal flange fits snugly into at least one longitudinal channel or groove located on the inner wall of the axial bore.

In operation, a hole is drilled between adjacent vertebrae. The frangible, tubular, outer-body portion containing the expansion cylinder is inserted into the hole. During insertion, the low-profile rib flattens, expanding out into the surrounding bone. Next, the expansion cylinder rotates through a ninety degree angle. As the flange moves out of the mating channel on the inner surface of the axial bore, the flange presses the outer-body portion tightly against the surrounding bone, thereby forcing the low profile rib even deeper into the bone. The expansion cylinder may further include an axial bore containing a bone graft material that has at least one aperture on its outer surface.

In yet another embodiment, the frangible, tubular, outer-body portion has an expansion cylinder slidably mounted within an axial bore. The expansion cylinder may include a bone graft material housed within its axial bore. The outer-body portion further includes at least one elastically-deformable high profile rib on its outer surface.

In operation, a hole is drilled between adjacent vertebrae and the implant is inserted into the hole. Rotation of the expansion cylinder through a ninety degree angle separates the outer-body portion, dislodging the high profile rib from its housing. The rib now extends into the bone to anchor the implant within the hole.

### BRIEF DESCRIPTION OF THE DRAWINGS

In order to better understand various exemplary embodiments, reference is made to the accompanying drawings, wherein:

FIG. 1 is a perspective view of an expandable, intervertebral implant comprising a locking rib positioned on the outer surface of a non-frangible, tubular, outer-body portion of the implant and a bone graft positioned within the axial bore of the outer-body portion;

FIG. 2 is a perspective view of an expandable, intervertebral implant comprising an expansion cylinder slidably disposed within the axial bore of a tubular, outer-body portion and a locking rib positioned on the outer surface of the non-frangible outer-body portion of the implant, and a bone graft positioned within the axial bore of the expansion cylinder;

FIG. 3 is a perspective view of an expandable, intervertebral implant comprising an expansion cylinder slidably disposed within the axial bore of a tubular, outer-body portion and a locking rib positioned on the outer surface of the frangible outer-body portion of the implant, and a bone graft positioned within the axial bore of the expansion cylinder;

FIG. 4 is a perspective view of the tubular, outer-body portion of FIG. 3;

FIG. 5 is a perspective view of the expansion cylinder of FIG. 2;

FIG. 6 is a perspective view of a low profile locking rib positioned on the outer-body portion of FIG. 1; and

FIG. 7 is a perspective view of a high profile locking rib positioned on the outer-body portion of FIG. 2.

### DETAILED DESCRIPTION OF THE PREFERRED EMBODIMENTS OF THE INVENTION

Referring now to the drawings, in which like numerals refer to like components or steps, there are disclosed broad aspects of various exemplary embodiments.

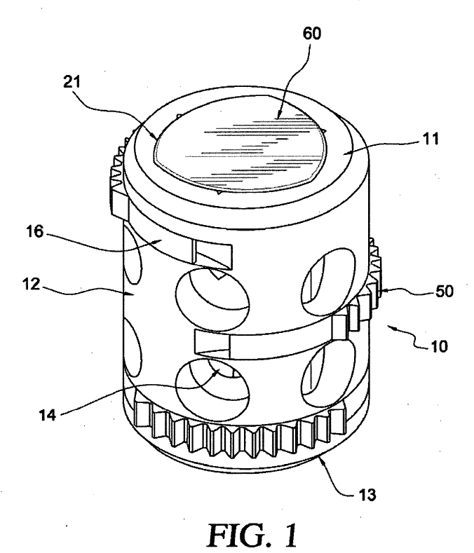

FIG. 1 is a perspective view of an expandable, intervertebral implant 10 comprising at least one low profile locking rib 50 positioned on the outer surface of a non-frangible, tubular, outer-body portion 12 of the implant and a bone graft positioned within an axial bore 21 of the outer-body portion 12. Elastically-deformable, low profile ribs 50 are positioned in rib housing 16 of outer-body portion 12. Implant 10 has a proximal end 11 and a distal end 13. The wall of outer-body portion 12 has a plurality of holes 14. A cylindrical axial bore 21 is coextensive with the length of outer-body portion 12. Axial bore 21 of outer-body portion 12 contains bone graft material 70, which may be bone chips or a suitable osteogenic material.

In order to use implant 10, a hole is first drilled between adjacent vertebrae in a chordate animal having a backbone in a direction substantially transverse to the axis of the spine. This hole is, centered between adjacent vertebrae. Outer-body portion 12 with low profile ribs 50 is inserted into the hole. Application of external pressure to distal ends 17, 18 of rib housing 16 depresses low profile locking ribs 50. After outer-body portion 12 is fully inserted, low profile locking ribs 50 flatten out, expanding into the surrounding bone. Implant 10 is now locked in position between adjacent vertebrae.

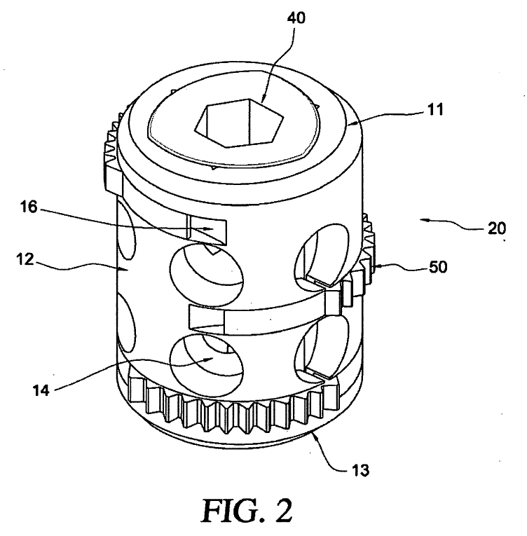

FIG. 2 is a perspective view of an expandable, interveriebral implant 20 comprising an expansion cylinder 40 slidably disposed within an, axial bore 21 of a tubular, outer-body portion 12 and at least one high profile locking rib 60 positioned on the outer surface of the non-frangible outer-body portion 12 of the implant 20, and a bone graft positioned within axial bore 21 of expansion cylinder 40. Implant 20 has a proximal end 11 and a distal end 13. As for implant 10, depicted in FIG. 1, the wall of outer-body portion 12 has a plurality of holes 14 and cylindrical axial bore 21 is coextensive with the length of outer-body portion 12.

In order to use implant 20, a hole is first drilled between adjacent vertebrae in a direction substantially transverse to the axis of the spine. As in FIG. 1, this hole is centered between adjacent vertebrae. Next, outer-body portion 12 with high profile locking ribs 60 is inserted into the hole. The outer diameter of expansion cylinder 40 is dimensioned to slidably fit within axial bore 21. Application of external pressure to distal ends 17, 18 of rib housing 16 depresses high profile locking ribs 60. Rotating cylinder 40 through a ninety degree angle then dislodges high profile ribs 60 from their housing 16.

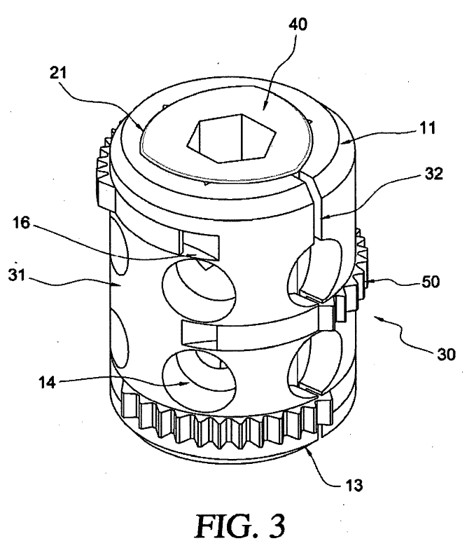

FIG. 3 is a perspective view of an expandable, intervertebral implant 30 comprising an expansion cylinder 40 slidably disposed within an axial bore 21 of a tubular, outer-body portion 31 and at least one low profile locking rib 50 positioned on the outer surface of the frangible outer-body portion. 31 of the implant 30, and a bone graft positioned Within axial bore 21 of expansion cylinder 40. Outer-body portion 31 has a frangible section 32. Implant 30 has a proximal end 11 and a distal end 13. In a structure similar to FIGS. 1 and 2 the wall of outer-body portion 31 has a plurality of holes 14 and axial bore 21 is coextensive with the length of outer-body portion 31.

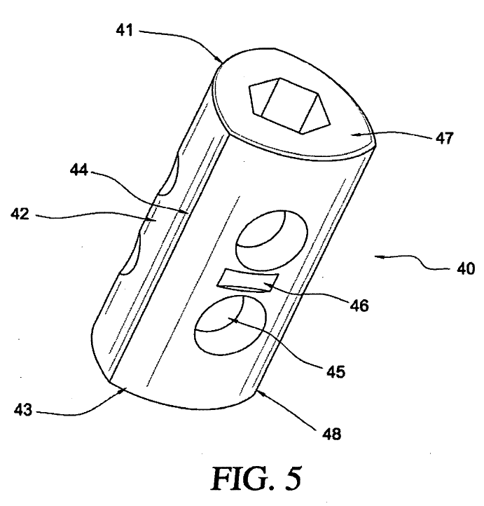

In order to use implant 30, a hole is first drilled between adjacent vertebrae in a direction substantially transverse to the axis of the spine. This hole is centered between adjacent vertebrae. Outer-body portion 31 with low profile locking ribs 50 is inserted into the hole. The outer diameter of expansion cylinder 40 is dimensioned to slidably fit within axial bore 21. Application of external pressure to distal ends 17, 18 of rib housing 16 depresses low profile locking ribs 50. The rotation of cylinder 40 through a ninety degree angle causes flanges, further described in FIG. 5, of expansion cylinder 40 to move out of grooves 19, 22 on the inner surface of axial bore 21. This motion elevates the tubular outer body, makes its surface press tightly against the surrounding bone, and forces low profile locking ribs 50 to dislodge from housing 16. Expansion cylinder 40 may further include an axial bore 21 having a plurality of holes in its outer surface that contains a bone graft material.

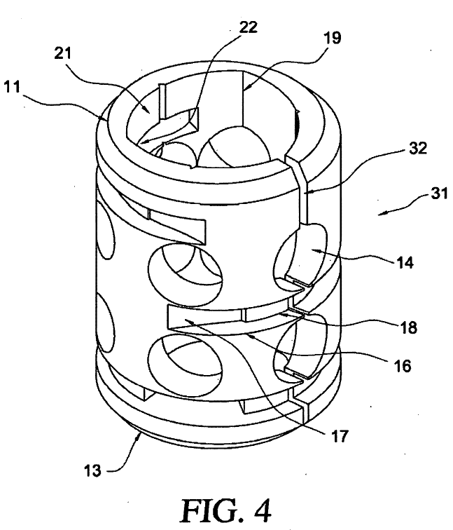

FIG. 4 is a perspective view of the tubular, outer-body portion of FIG. 3 without expansion cylinder 40 and high profile locking rib 60.

FIG. 5 is a perspective view of expansion cylinder 40 of FIG. 2. Referring to FIG. 5, expansion cylinder 40 has a proximal end 41, an elongated surface 42, a distal end 43, and a proximal surface 47. Cylinder 40 fits snugly into axial bore 21 of implants 20, 30,just as flanges 44, 48 of cylinder 40 fit snugly into grooves 19, 22 on the inner surface of axial bore 21. Proximal surface 47 has a ratcheting construction to facilitate rotation of cylinder 40 inside axial bore 21 of implants 20, 30. Apertures 45, 46 provide a plurality of conduits between cylinder 40 and implants 20, 30 to allow bone graft, 70 to access the surrounding bone. For implant 30, cylinder 40 separates the outer-body portion, elevates it, and dislodges high profile locking rib 60 from housing 16 in order to anchor implant 30 within the hole.

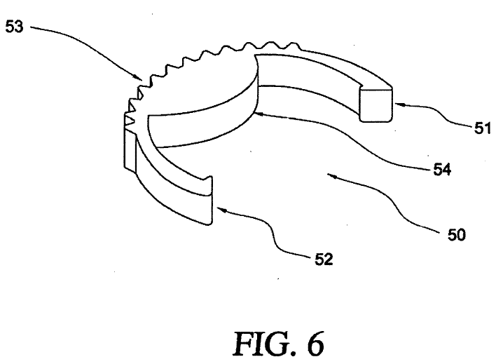

FIG. 6 is a perspective view of low profile locking rib 50 positioned on the outer-body portion of FIG. 1. Low profile locking rib 50 is elastically deformable and has locking feet 51, 52, a short spike 53, and a distal, flat surface 54. As implant 10 advances into the bone, low profile locking rib 50 deforms inwardly toward distal surface 54. When implant 10 is fully inserted into the hole, low profile locking rib 50 is flattened, driving short spike 53 of low profile locking rib 50 into the surrounding bone, thereby anchoring implant 10 within the hole.

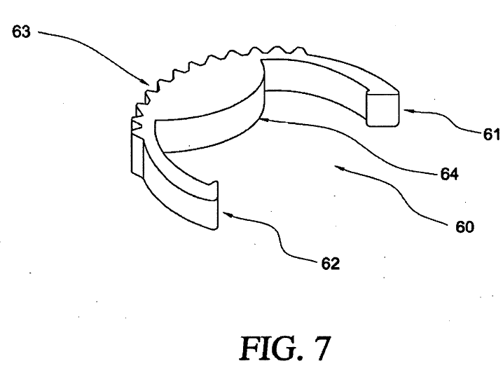

FIG. 7 is a perspective view of high profile locking rib 60 positioned on the outer-body portion of FIG. 2. High profile locking rib 60 has locking feet 61, 62, a short spike 63, and a distal, bow-shaped surface 64. The outer diameter of expansion cylinder 40 is dimensioned to slidably fit within axial bore 21 of implants 20, 30. Rotating cylinder 40 clockwise through a ninety degree angle will allow flanges 44, 48 of expansion cylinder 40 to move out of grooves 19, 22 on the inner surface of axial bore 21. Subsequently, flanges 44, 48 will fully contact distal surface 64, thereby dislodging high profile locking ribs 60 from housing 16. As ribs 60 extend outwardly into the surrounding bone, they will anchor implants 20, 30 within the hole. Short spike 63 extends outwardly away from the first outer surface, pressing even deeper into the bone and locking the entire device into position.

Low profile locking rib 50, high profile locking rib 60, and short spikes 54, 64 are formed out of polymer blends of glycolide and/or lactide homopolymer, copolymer and/or glycolide, lactide copolymer and polycaprolactone copolymers, and/or copolymers of glycolide, lactide, poly (L-lactide-co-DL-lactide), caprolactone, polyorthoesters, polydioxanone, trimethylene carbonate, polyethylene oxide, or any other bioabsorbable material. A pseudoelastic shape memory alloy may also be used to fabricate locking ribs 50, 60. One such pseudoelastic shape memory alloy might be a nickel titanium alloy such as Nitinol™, which is available from Flexmedics of Minneapolis, Minn. The use of such a material, in combination with the normal orientation of locking ribs 50, 60 relative to the anchor body, permits locking ribs 50, 60 to deflect inwardly at first but later spring back resiliently toward their normal, outwardly projecting position. Expansion cylinder 40, ribs 50, 60, and outer-body portion 12, 31 may be made from the same material or from different materials, selected from the group consisting of a bioabsorbable polymer, a ceramic, a pseudoelastic shape memory alloy, titanium, stainless steel, and a cobalt-chromium alloy.

It should be apparent that other changes and modifications can be made without departing from the spirit and scope of the invention. For example, axial elevation of the implant may be performed by other means such as conically-shaped cylinders, screw, nail, wedge-driven expanders, or any other expansion driven design. The outer-body portion can expand partially, expand fully, or remain undeformed while the expansion cylinder advances into the axial bore in a distal direction. The outer-body portion may have a polygonal cross-sectional profile, such as a hexagonal cross-sectional profile.

Although the various exemplary embodiments have been described in detail with particular reference to certain exemplary aspects thereof, it should be understood that the invention is capable of other embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

What is claimed is:

1. An expandable, intervertebral implant for implantation within a hole drilled between adjacent vertebrae in the spine of an animal, thereafter enabling the adjacent vertebrae to fuse to one another, comprising:
an elongate, non-frangible, tubular member having an axial bore and a cylindrical outer surface dimensioned to fit snugly within said drilled hole;
a bone graft material disposed within said axial bore;
at least one hole in said cylindrical outer surface extending inwardly toward said axial bore; and
at least one elastically-deformable rib on said cylindrical outer surface.

2. The implant of claim 1, wherein said at least one rib is made from at least one material selected from the group consisting of a bioabsorbable polymer, a ceramic, a pseudoelastic shape memory alloy, titanium, stainless steel, and a cobalt-chromium alloy.

3. An expandable, intervertebral implant for implantation within a hole drilled between adjacent vertebrae in the spine of an animal, thereafter enabling the adjacent vertebrae to fuse to one another, comprising:
a non-frangible, tubular, outer-body portion having a proximal end, a distal end, and an elongate body portion with a first axial bore, said outer-body portion having a generally cylindrical first outer surface with at least one first aperture;
an elongate, expansion cylinder slidably disposed within said first axial bore, said expansion cylinder having a second axial bore and a generally cylindrical second outer surface with at least one second aperture;
a bone graft material disposed within said second axial bore;
at least one locking rib disposed on said first outer surface wherein when said expansion cylinder rotates within said first axial bore, said at least one locking rib dislodges and penetrates into the surrounding bone; and
at least one conduit in said second outer surface between said second outer surface and said second axial bore.

4. The implant of claim 3, wherein said first outer surface has a triangular cross-section when viewed in the direction of the axial bore.

5. The implant of claim 4, wherein said second outer surface has a triangular cross-section when viewed in the direction of the axial bore.

6. The implant of claim 3, wherein said first outer surface has an elliptical cross-section when viewed in the direction of the axial bore.

7. The implant of claim 6, wherein said second outer surface has an elliptical cross-section when viewed in the direction of the axial bore.

8. The implant of claim 3, wherein said at least one rib is made from at least one material selected from the group consisting of a bioabsorbable polymer, a ceramic, a pseudoelastic shape memory alloy, titanium, stainless steel, and a cobalt-chromium alloy.

9. An expandable, intervertebral implant for implantation within a hole drilled between adjacent vertebrae in the spine of an animal, thereafter enabling the adjacent vertebrae to fuse to one another, comprising:
a tubular, outer-body portion comprising a non-frangible cylinder having a proximal end, a distal end, and a first axial bore, said outer-body portion having a generally cylindrical first outer surface with at least one first aperture;
an elongate, expansion cylinder slidably disposed within said first axial bore, said expansion cylinder having a second axial bore and a generally cylindrical second outer surface with at least one second aperture;
at least one high profile locking rib disposed on said first outer surface, wherein when said expansion cylinder rotates within said first axial bore, said at least one high profile locking rib dislodges and penetrates into the surrounding bone;
a bone graft material disposed within said second axial bore; and
at least one conduit in said second outer surface between said second outer surface and said second axial bore.

10. The implant of claim 9, wherein said at least one high profile locking rib is made from at least one material selected from the group consisting of a bioabsorbable polymer, a ceramic, a pseudoelastic shape memory alloy, titanium, stainless steel, and a cobalt-chromium alloy.

11. The implant of claim 9, wherein said first outer surface has a triangular cross-section when viewed in the direction of the axial bore.

12. The implant of claim 9, wherein said second outer surface has a triangular cross-section when viewed in the direction of the axial bore.

13. The implant of claim 9, wherein said first outer surface has an elliptical cross-section when viewed in the direction of the axial bore.

14. The implant of claim 9, wherein said second outer surface has an elliptical cross-section when viewed in the direction of the axial bore.

15. An expandable, intervertebral implant for implantation within a hole drilled between adjacent vertebrae in the spine of an animal, thereafter enabling the adjacent vertebrae to fuse to one another, comprising:
a tubular, outer-body portion comprising a frangible cylinder, said cylinder having at least one longitudinal slit, a proximal end, a distal end, and a first axial bore, wherein said outer-body portion has a generally cylindrical first outer surface with at least one first aperture;
an elongate, expansion cylinder slidably disposed within said first axial bore, said expansion cylinder having a second axial bore and a generally cylindrical second outer surface with at least one second aperture;
a bone graft material disposed within said second axial bore;
at least one high profile rib disposed on said first outer surface, wherein, when said expansion cylinder rotates within said first axial bore, said at least one high profile locking rib dislodges and penetrates into the surrounding bone and elevates said outer-body portion against opposing surfaces to press tightly against the surrounding bone; and
at least one conduit in said second outer surface between said second outer surface and said second axial bore.

16. The implant of claim 15, wherein said at least one high profile rib is made from at least one material selected from the group consisting of a bioabsorbable polymer, a ceramic, a pseudoelastic shape memory alloy, titanium, stainless steel, and a cobalt-chromium alloy.
